(19) United States
(12) Patent Application Publication
(10) Pub. No.: US 2008/0254486 A1
(43) Pub. Date: Oct. 16, 2008

(54) Prion Sensors for Diagnosis of Transmissible Spongiform Encephalopathy or for Detection of Prions, and Use Thereof

(75) Inventors: Gordon Hayward, Kitchener (CA); Warren Stiver, Guelph (CA); John Ellis, Guelph (CA); Vicky Wong, Guelph (CA)

Correspondence Address: BENNETT JONES;C/O MS ROSEANN CALDWELL, 4500 BANKERS HALL EAST, 855 - 2ND STREET, SW, CALGARY, AB, T2P 4K7, CA

(73) Assignee: UNIVERSITY OF GUELPH, Guelph, ON (CA)

(21) Appl. No.: 11/720,962

(22) PCT Filed: Dec. 9, 2005
(86) PCT No.: PCT/CA05/01867

§ 371 Date: Aug. 29, 2007

Related U.S. Application Data
(60) Provisional application No. 60/593,150, filed on Dec. 15, 2004.

(51) Int. Cl. G01N 33/566; C12M 1/34

(52) U.S. Cl. 435/7.8; 435/288.7; 435/287.1

(57) ABSTRACT

Disclosed are prion sensors that may be used as a diagnostic tool to diagnose TSE, or to detect PrP^{Sc} molecules, in biological and environmental samples, and methods for using these sensors. The prion sensor may comprise an acoustic sensor, such as a TSM sensor, coated with PrP^{C} molecules, whose resonance characteristics change when contacted with a sample from an animal with TSE, or a sample comprising PrP^{Sc} molecules. Alternatively the prion sensor may comprise an optical sensor, such as an SPR sensor, coated with PrP^{C} molecules, whose optical characteristics change when contacted with a sample from an animal with TSE, or a sample comprising PrP^{Sc} molecules. These changes provide both rapid detection and a quantitative assay for diagnosing TSE or for detecting PrP^{Sc} molecules.

### FIELD

This invention relates to prion sensors that may be used as a tool to diagnose TSE, or to detect PrP^{Sc}, in biological and environmental samples, and methods for using these prion sensors.

### BACKGROUND

Prions are the infectious pathogens that cause central nervous system transmissible spongiform encephalopathies (TSE's) in animals, including: Scrapie in sheep; Chronic Wasting Disease (CWD) in deer; Bovine Spongiform Encephalopathy (BSE or “mad cow” disease) in cattle; transmissible mink encephalopathy (TME) in mink; feline spongiform encephalopathy (FSE) in cats; and kuru, Creutzfeldt-Jakob disease (CJD), Gerstmann-Strassler-Scheinker disease (GSS) and fatal familial insomnia (FFI) in humans. These diseases are transmitted by agents called prions, which are hypothesized to be proteinaceous only, containing no genetic material (Prusiner, 1996).

Protein prions (PrP) are sialoglycoproteins normally found on the outer surfaces of neurons, and appear to exist in two forms, which differ only in their folding or conformation. One form, cellular prion protein (PrP^{C}), is found in the normal tissue of mammals, and the other form, scrapie prion protein (PrP^{Sc}) is the infectious and pathogenic agent, found in diseased tissue. Although PrP^{C} and PrP^{Sc} have the same amino acid sequence (Prusiner, 1998), PrP^{C} has 42% of its peptides folded in an α-helix configuration with little (3%) in the β-sheet form while PrP^{Sc} has 30% in the α-helix form and 43% in the β-sheet form. The change to the β-sheet form appears to confer resistance to digestion by protease enzymes where the resistant fragment, consisting of 50% β-sheet form, retains its infective capacity. The refolding of the normal protein into the β-sheet form is, therefore, the primary lesion of prion disease (Pan et al., 1993).

One leading hypothesis is that prion diseases result when the infectious agent PrP^{Sc} infects a susceptible animal and, acting as a template, causes the normal PrP^{C} to refold into PrP^{Sc} (Collinge, 2001), which process then repeats itself as the newly formed PrP^{Sc} causes additional PrP^{C} to refold. The refolding is hypothesized to occur through one of two mechanisms, although neither has been conclusively proven (Borman, 1998). The first mechanism involves the formation of a dimer wherein one PrP^{C}, which is water soluble, attaches to a PrP^{Sc}, refolds and dissociates into two PrP^{Sc} units. In the second mechanism, soluble PrP^{C} attaches to an insoluble PrP^{Sc} aggregate, refolds and remains attached, adding to the aggregate. Evidence for the latter mechanism is the increase in the PrP^{C} to PrP^{Sc} conversion rate when the aggregates are broken up by sonication (Saborio et al., 2001). It has also been postulated that a chaperone protein which catalyses the refolding may be involved (Prusiner, 1998).

Although many cases of CJD arise spontaneously in humans, persons that have received growth hormone from human pituitaries, donated corneas or donated dura mater grafts have been infected, and it is hypothesized that an atypical new variant (nvCJD) is caused by the transmission of bovine prions from BSE infected cows to humans by the consumption of beef products. Recent evidence supports this hypothesis (Bruce, 2003).

There are several assay systems currently in use to detect prions. Each is less than satisfactory for several-reasons. The most sensitive test is the bioassay in which a test animal or test animals are inoculated with samples of the suspect material. In the diagnosis of disease, this suspect material is tissue taken from the suspected animal or human. This is unacceptably invasive. The test animal(s) are then allowed to develop the disease. TSE's have a long incubation period. For example in sheep and cattle it can take months from the time the animal becomes infected until it first shows disease signs. See for instance U.S. Pat. No. 6,008,435, which discloses a transgenic mouse that can be used for monitoring BSE in an assay which takes 250-350 days to provide a result. Infected animals and humans do not have a disease-specific immune response, nor consistent biochemical, haematological and gross pathological abnormalities. The test animals are euthanized and their brain tissue is examined post mortem.

The examination of the brain samples obtained post mortem can be done histologically to observe the microscopic holes characteristic of the diseases in its late stage. The PrP^{Sc} protein itself may be observed directly by electron microscopy where the agglomerated form is visualized as Scrapie associated fibrils extracted from the infected brain tissue. This technique is very expensive and time consuming. A more sensitive technique, immunohistochemical examination, which can be applied earlier in the disease process, involves staining the PrP^{Sc} on the nerve cell walls with an antibody specific for a small region of the PrP molecule, and observing this through the microscope. The use of these antibodies is complicated by the fact that the same region is present on both PrP^{C} and PrP^{Sc}; therefore the samples require pretreatment with protease K to destroy the normal PrP^{C} (Borman, 2001). U.S. Pat. No. 6,165,784 describes antibodies for the detection of PrP^{Sc}.

This type of antibody can also be used to determine the presence of prions in homogenized samples. This test is an ELISA (Enzyme Linked Immuno-Sorbent Assay) where one anti-PrP antibody, attached to a surface, binds the PrP. After washing, another enzyme-linked anti-PrP antibody binds to the attached PrP. This enzyme then catalyses a colour development reaction where the intensity of the colour is proportional to the amount of PrP in the sample. Again, the antibodies respond to both PrP^{C} and PrP^{Sc}, so a pre-digestion with protease K is required to eliminate the normal PrP^{C}. This digestion step adds to the complexity, time duration and cost of the assay and considerably dilutes the sample, reducing the sensitivity of the test. This system is commercially available, for example as the Platelia system (BioRad, Hercules, Calif.).

To further increase the sensitivity of the antibody assay, the Western Blot technique is used. After the protease K digestion, the PrP^{Sc} is denatured to render it soluble, purified by gel electrophoresis, transferred to a test membrane and stained by an anti-PrP antibody attached to an indicating enzyme. This enzyme catalyses a chemiluminescent reaction detected photographically. Although more sensitive, the protease K digestion is still required and the test is more elaborate than the ELISA method. It is also commercially available, for example from Prionics AG (Schlieren, Switzerland).

There remains a need for an assay system that is rapid, sensitive, reliable and technically simple. Preferably, this assay would diagnose TSE infection and/or detect prions in tissue from live animals and humans, would be relatively non-invasive and would be sensitive enough for diagnosis at a preclinical disease stage. In particular, an assay for the routine monitoring of both live and dead cattle and sheep would be useful to reduce the spread of the disease, because these animals are used for human consumption. An assay that can test suspect animals quickly could avoid the mass slaughter of uninfected animals. Additionally, a method of prion detection based on their infectious capability would be superior to a method of detection based on the presence or absence of an immunologically reactive prion fragment.

There are several sensor systems that have been shown to respond to the binding and subsequent molecular changes of biological molecules. These include Thickness Shear Mode (TSM) acoustic devices and Surface Plasmon Resonance (SPR) Systems. The acoustic response in a TSM sensor occurs as the molecules bound to the sensor surface change, in turn changing the mechanical resonance properties of the TSM sensor, which vibrates at a high frequency. SPR is an optical technique where changes in molecules bound to one surface of a thin metal film, change the reflection of light from the other side of the metal film. Both the acoustic resonance and the optical reflection respond to subtle changes in the molecules binding to the respective sensor. These responses are easily measured.

The TSM is a device that generates acoustic vibrations from an electrical signal, typically through the piezoelectric effect, and uses these vibrations to detect and/or quantify particular chemical or biochemical substances (the analyte) present in a sample surrounding the sensor, Acoustic energy is stored and dissipated both in the sensor itself, and through interfacial coupling, in a surrounding liquid medium. By coating the sensor with one or more layers of a substance (the receptor) that interacts with the analyte, the energy storage and transfer processes change when interaction occurs. These change the acoustic resonance of the sensor, which can be observed by measuring the resonant frequency and electrical impedance of the sensor [Cavic et al. (1997); Ferrante et al., (1994); Hayward and Thompson (1998); Su and Thompson (1996)].

There are several mechanisms whereby a TSM sensor immersed in a sample responds to a chemical change in the receptor coated onto its surface. Surface mass deposition occurs when the analyte binds to the receptor, increasing the storage of acoustic energy through the inertia of the added mass. Acoustic energy may also be stored through the elastic deformation of the surface coating, when this coating is thick. The elasticity of the receptor coating may also change when the analyte binds to it. Viscous loading occurs when acoustic energy is transferred to the liquid surrounding the sensor. Some of the energy stored by the inertia of the liquid moving with the sensor is transferred back to the sensor, but acoustic energy is also dissipated by internal friction within the liquid. These energy storage and dissipation modes determine the resonant characteristics of the sensor, which can easily be measured electrically. The inertial and elastic acoustic energy storage processes affect the resonant frequency while the acoustic energy dissipation process appears as an electrical resistance. Since this resistance is due to a mechanical process, it is referred to as the motional resistance. Examples of acoustic sensors are described, for example, in U.S. Pat. Nos. 5,374,521 and 5,658,732

WO 01/23892 A1 discloses a process for sensing biological or chemical change in molecules that is based on measurements of phenomena based on imperfect coupling between the sensor surface and a liquid surrounding the sensor. The nature of this coupling determines the strength of the viscous loading and elastic effects, depending on such parameters as the surface free energy and the molecular conformation of the receptor coating. These molecular parameters are very sensitive to chemical changes at the surface and therefore acoustic coupling provides a novel sensing mechanism.

A SPR device is capable of detecting changes in a film of molecules attached to a sensor surface. An optical beam, created by a laser or other light source, reflects from one side of a thin metal film. The reflection from one side of the metal film produces an electric field which extends for a short distance beyond the other side of the metal film. When this field extends into a surface film, for example a layer of attached protein molecules, changes in the attached protein molecules alter the field, which in turn changes the reflection angle of the light beam. Therefore, a SPR device can measure refractive index changes that are induced by interaction of the attached protein molecules with an analyte in solution.

Although the TSM and SPR detection systems are based on different physical principles, they give very similar results for a variety of surface films (Bailey et al., 2002). Therefore, either instrument may be suitable for the method disclosed herein. Laschitsch et al. (2000) point out that the response of the two instruments depends on the change in contrast as the molecular film changes, acoustic contrast for the TSM device and optical contrast for SPR instrument. They show that the change in acoustic contrast is higher than the changes in optical contrast, therefore the TSM device may be preferred for the method disclosed herein. However, although the TSM device may be preferred, the methods may be practiced with other acoustic sensors or with optical sensors.

### SUMMARY

The applicants have shown that the PrP^{C} molecule may be used as a sensing molecule, in an acoustic or optical measuring device, to diagnose TSE, or to detect PrP^{Sc} molecules, in a sample. Therefore, disclosed herein is a prion sensor, and a method, useful for diagnosing TSE infection, or for detecting PrP^{Sc} molecules, in a sample. The method is rapid, sensitive and technically simple. This assay diagnoses TSE infection, or detects PrP^{Sc} molecules, in fluid or tissue samples taken from a mammal, or from environmental samples. In one aspect the assay system is quantitative.

In one aspect the invention is a method of diagnosing TSE, or of detecting PrP^{Sc} molecules, in a sample, which method comprises:

(a) attaching PrP^{C} molecules to a surface of a sensor directly, or via a linking layer, to form a sensing layer;
(b) contacting the sensing layer with the sample;
(c) measuring an acoustic or optical response in the sensor, and
(d) interpreting the response of the sensor, to diagnose TSE infection, or to detect PrP^{Sc} molecules, in the sample.

In one embodiment the sensor is an acoustic sensor, and the acoustic response in the sensor is determined by:

(a) inducing oscillating motion at the acoustic resonant frequency of the sensor;
(b) measuring this resonant frequency, or alternatively measuring the energy loss as:
(i) a motional resistance, or
(ii) a dissipation factor, and
   (c) interpreting the measurement of resonant frequency, the motional resistance and/or the dissipation factor, to diagnose TSE, or to detect PrP^{Sc} molecules in the sample.

In one embodiment the acoustic sensor is a TSM sensor.

In another embodiment, the sensor is an optical sensor, and the optical response in the sensor is determined by:

(a) applying an optical beam to the sensor;
(b) measuring the refraction and/or reflection characteristics of the sensor, and;
(c) interpreting the measurement of the refraction and/or reflection characteristics to diagnose TSE, or to detect PrP^{Sc} molecules in the sample.

In one embodiment the optical sensor is an SPR sensor.

The step of measuring the acoustic or optical response in the sensor may be performed while the sensing layer is in contact with the sample.

A chaperone protein or a tissue extract comprising a chaperone protein may be added to the sample. In one embodiment, more than one layer of PrP^{C} molecules is attached to the surface of the sensor, to form the sensing layer. In one embodiment the method is practiced with samples from a mammal. The mammal may be selected from a group that includes: sheep, deer, cow, human, mink, hamster, mouse, goat and cat. In one embodiment the sample is comprised of an extract of a tissue from a mammal. The tissue may be selected from a group that includes: tonsil, eyelid, brain, rectal and lymphatic. In one embodiment the tissue is brain. In one embodiment the sample is comprised of bodily fluid from a mammal. The bodily fluid may be selected from a group that includes: blood, serum, urine and cerebrospinal fluid. In one embodiment the bodily fluid is urine. In another embodiment the sample is excrement. In another embodiment the sample is from an artificial tissue culture or is an environmental sample.

In another aspect the invention is the use of PrP^{C} as a sensing molecule in an acoustic or optical measuring device that can detect molecular changes in the sensing molecule, to diagnose TSE, or to detect PrP^{Sc} molecules, in a sample. The PrP^{C} may be isolated from healthy animal tissue, produced by a recombinant host organism or artificially synthesized. The acoustic device may be a thickness shear piezoelectric oscillating molecular sensing device. The optical device may be a SPR device.

In another aspect the invention is a prion sensor comprising an acoustic sensor and a layer of PrP^{C} molecules attached directly or indirectly to a surface of the acoustic sensor. In one embodiment the acoustic sensor is a TSM sensor. In another aspect, the invention is a prion sensor comprising PrP^{C} molecules attached directly or indirectly to a surface of an optical sensor. In one embodiment the optical sensor is an SPR sensor.

In another aspect the invention is a method of quantitating the amount of PrP^{Sc} in a sample, which method comprises practicing the method disclosed above and interpreting the response in the series resonant frequency, the motional resistance, the acoustic dissipation factor, or the refraction and/or reflection of the optical beam, to determine the amount of PrP^{Sc} in the sample. Fluid or tissue samples that are collected from subject mammals will contain different concentrations of PrP^{Sc} molecule, depending on the type of sample. For example, tonsil tissue will have higher concentrations of PrP^{Sc} molecules than blood. Alternatively, depending on how advanced the disease state is, the same fluids or tissues from different subject mammals may have very different concentrations of PrP^{Sc} molecules.

In another aspect the invention is a method of determining cross-species and cross-genotype infection susceptibility and efficiency. In this method the PrP^{C} molecules are from a species or genotype that is different than the species or genotype of the sample or PrP^{Sc} molecules.

### BRIEF DESCRIPTION OF THE DRAWINGS

FIG. 1: An embodiment of the prion sensor (10) which embodiment is useful in a TSM device. This embodiment comprises a sensor (20) that is made of a crystal wafer (1) and gold electrodes (3 and 9) attached to the wafer on either side. Electrical connections may be made at the extended electrodes on the wafer edge. Optional binding layer (5) and a sensing layer (6) are attached to the top electrode (3).

A glass vial (4) glued to the sensor using flexible silicone cement (2), may hold the sample (8) to be analysed. The sensing response occurs as reactants (7) in the sample are bound by, or otherwise interact with, the sensing layer (6).

FIG. 2: An embodiment of the prion sensor (10a) useful in a SPR device. The prion sensor (10a) comprises a sensor (20a) that is a layer of metal (11) attached to a prism or diffraction grating (14), an optional binding layer (5a) and a sensing layer (6a) to one side.

The sensing response occurs as reactants (7a) in solution are bound by, or otherwise interact with, the sensing layer (6a). The interaction of the reactants (7a) with the sensing layer (6a) is monitored by an optical detector (12) located on the opposite side of the sensor surface from the sensing layer (6a), which detects a response in the refraction of an optical beam (13) as it is reflected from the surface of the sensor.

FIG. 3: Frequency response of a TSM sensor with bound sheep PrP^{C} molecules, to normal sheep brain homogenates and homogenates from sheep with Scrapie. The difference is clearly measured.

FIG. 4: The frequency difference, calculated from the observed data and a simple first order model for infected and normal samples, more clearly shows the infection.

FIG. 5: The peak height from the frequency difference curves obtained from brain tissue sample of sheep with Scrapie diluted with different amounts of normal sheep brain tissue shows that the result observed is logarithmically proportional to the PrP^{Sc} concentration in the sample.

FIG. 6: The frequency response of a TSM sensor with bound sheep PrP^{C} molecules, to brain homogenate samples from normal elk and from elk with Chronic Wasting Disease, calculated as the difference between the observed data and a simple first order model, clearly shows the infection.

FIG. 7: The height of the peaks and the peak time shows the difference between brain homogenate samples from sheep with Scrapie and brain homogenate samples from elk with Chronic Wasting Disease, analysed using immobilized sheep PrP^{C}.

FIG. 8: Frequency response of a TSM sensor with bound sheep PrP^{C} molecules, to urine from normal sheep and from sheep with Scrapie. The difference is clearly measured.

### DETAILED DESCRIPTION OF THE PREFERRED EMBODIMENTS

Unless defined otherwise, technical and scientific terms used herein have the same meaning as commonly understood by one of ordinary skill in the art to which this invention pertains.

As used herein, PrP^{C} or “PrP^{C} molecule” refers to the non-infectious protein prion molecule as it is folded in the brain and other tissue of a TSE-free mammal of any kind. This term also includes any fragment of a PrP^{C} molecule that can be used to form a sensing layer, said sensing layer being used to diagnose TSE or detect PrP^{Sc}, as contemplated by the method disclosed herein. A “TSE-free” mammal is an animal that does not have a transmissible spongiform encephalopathy, whether symptomatic or asymptomatic.

PrP^{Sc} or “PrP^{Sc} molecule”, or “prion” refers to the infectious protein prion molecule, and may include any infectious fragment of a PrP^{Sc} molecule. These terms include infectious protein prions that would cause any type of transmissible spongiform encephalopathy, including Scrapie, BSE, TME, FSE, Kuru, CJD, GSS and FFI and any other as yet unknown TSE in a mammal.

A “TSE-infected” or “TSE-diseased” mammal is an animal, including humans, that has a transmissible spongiform encephalopathy, whether symptomatic or asymptomatic.

“Diagnosis of TSE” or “diagnosing TSE” means determining whether an animal is TSE-infected or TSE-diseased, whether symptomatic or asymptomatic, and includes testing of a sample from an animal.

The prion sensor (10) disclosed herein is comprised of a sensor (20) to which has been attached a sensing layer (6) that comprises PrP^{C} molecules. The sensor (20) may be an acoustic sensor or an optical sensor. Although the preferred acoustic sensor is a TSM sensor, any type of acoustic resonating device may be used including: a Bulk Acoustic Wave Transverse Shear Mode Resonator, a Thin Rod Acoustic Wave Sensor, a Surface Acoustic Wave Sensor, a Surface Acoustic Transverse Wave Sensor, a Shear Horizontal Acoustic Plate Mode Sensor, or a Flexural Plate Wave Sensor. The acoustic resonator may be driven piezoelectrically, electrically or magnetically with or without attached electrodes. Examples of piezoelectric crystals suitable for use herein include quartz, lithium tantalite or niobate, oriented zinc oxide and aluminium nitride.

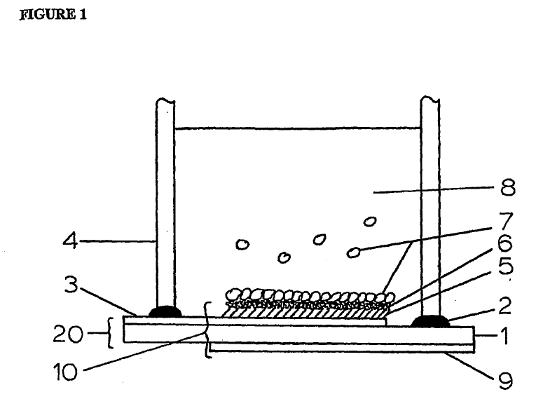

FIG. 1 shows an embodiment of the prion sensor (10) that may be used in a thickness shear mode (TSM) piezoelectric oscillating molecular sensing device. In this embodiment, the prion sensor (10) comprises a TSM sensor (20) and a sensing layer (6). The TSM sensor (20) may be made from a commercially available quartz wafer (1) or other piezoelectric crystal (e.g., lithium tantalite or niobate, oriented zinc oxide, aluminium nitride) with bound electrodes (3) and (9) made of gold or some other metal, on either side of the wafer. The edges of the crystal wafer may be provided with contacts to the electrical system.

Top electrode (3) may be coated with a linking layer (5) that functions to couple the sensing layer (6) to the surface of electrode (3). Therefore, the linking layer (5) may be a molecule or combination of molecules that bind to both the electrode and to the sensing layer. The linking layer may not be needed if the sensing layer (6) can be attached directly to the electrode, for example by adsorption.

Attached to the quartz wafer, for example with silicone cement (2), is a glass vial (4) to contain the sample solution or suspension. This vial may be replaced by a flow-through cell or other means of contacting the sample solution or suspension and the sensing layer.

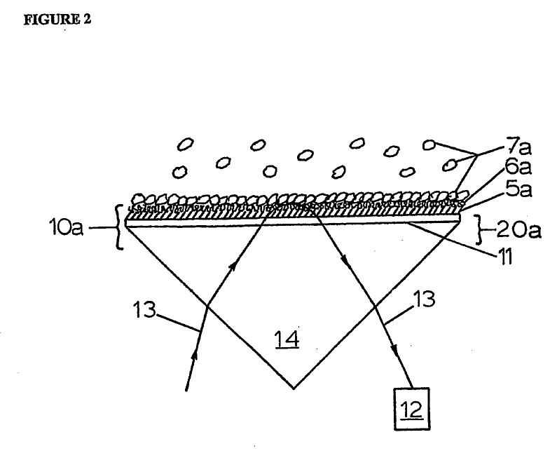

FIG. 2 shows another embodiment of the prion sensor (10a) that may be used in a SPR device. This embodiment comprises a SPR sensor (20a) that is a layer of metal (11) and a prism (14), an optional binding layer (5a) and a sensing layer (6a) attached to one side of the layer of metal (11) For immobilizing the sensing layer (6a) onto the surface of the SPR sensor (20a), similar means may be used as described above. The sensing response occurs as reactants (7a) in solution are bound by, or otherwise interact with, the sensing layer (6a). The interaction of the reactants (7a) with the sensing layer (6a) is monitored by an optical detector (12) located on the opposite side of the SPR sensor surface, which detects a change in the refraction and/or reflection of an optical beam (13).

Sensing layer (6) comprises PrP^{C} molecules, optionally in conjunction with other molecules as well. Thiol linking molecules (e.g., U.S. Pat. No. 5,834,224) are one means of attaching the sensing layer (6) to the gold electrode of the above-mentioned commercially available sensors. However, those of skill are aware that many other molecules or methods for the immobilization of proteins onto such surfaces are known and may be used according to the method disclosed herein. For example, silane reagents or neutravidin-biotin may be used.

Sensing layer (6) is bound to a surface of the sensor (20). In one embodiment, this surface may be an electrode. In another embodiment, this surface may be the surface of a piezoelectric crystal. In yet another embodiment, this surface may be the surface of the metal layer of an SPR sensor.

Linking layer (5) functions to couple sensing layer (6) to the surface of the sensor (20). Therefore, linking layer (5) may be a molecule or combination of molecules that bind to both the surface of the sensor and to the sensing layer. The linking layer may not be needed if the sensing layer (6) can be attached directly to the sensor surface, for example by adsorption.

Sensing layer (6) comprises PrP^{C} molecules that may be isolated and/or purified from healthy tissue, PrP^{C} molecules that may be produced by a recombinant host organism, synthetic PrP^{C} molecules, or PrP^{C} molecules from any other source. The PrP^{C} molecules may be from a species that is the same as the species from which the sample that is being analysed was obtained, in any particular assay. However, as is known, PrP^{C} molecules in different mammalian species are structurally similar. Therefore a PrP^{C} molecule from one mammalian species may be used to diagnose TSE, or to detect the PrP^{Sc} molecule, in a sample from another mammalian species. For instance, it is hypothesized that nvCJD is the result of the infection of humans by tainted beef, and therefore human and bovine PrP^{C} molecules are likely structurally similar. In the method disclosed herein therefore, BSE infection may be diagnosed, or bovine PrP^{Sc} molecules may be detected, by immobilizing the human PrP^{C} molecule to the sensor (20). In some instances the assay for a TSE, or for PrP^{Sc} molecules from a particular mammalian species, may prove to be more sensitive, or more rapid, if the immobilized PrP^{C} molecule is from a different species. This may be the case, for instance, if the PrP^{Sc} molecule is particularly efficient at causing a conformational change in the cross-species PrP^{C} molecule.

In one embodiment, the sensing layer may comprise several layers of PrP^{C} molecules, which may be added to the surface of the sensor by using a cross-linking agent. This may increase the amount of sample PrP^{Sc} that may be bound or the magnitude of the sensor response, thereby increasing the sensitivity of the method.

The response of an acoustic sensor (20), such as a TSM sensor, may be determined by measuring a change in the sensor acoustic resonance using a phase-locked oscillator, for example that produced by Maxtek, Inc. (Santa Fe Springs, Calif.). In particular, this oscillator seeks the series resonant frequency of a TSM sensor and measures the current through the TSM sensor to determine the motional resistance. The frequency chosen may be about 9 MHz, but this value is not critical. Another commonly used measurement system is based on impedance analysis. Here, impedance measurements are carried out by applying an electrical signal of known frequency and voltage to the sensor and measuring the current through the sensor to determine impedance at the known frequency. From these data, the series resonant frequency and the corresponding motional resistance may be obtained (Kipling and Thompson, 1990). A third system applies a pulse to start the oscillation and measures the frequency and magnitude of the decaying response. The resonant frequency and motional resistance may be calculated from these measurements (Q-Sense AB, Västra Frölunda, Sweden).

In another embodiment, the response of an optical sensor (20a), such as an SPR sensor, to the reactants in the sample may be determined by a using an SPR device. Here, an optical beam passes through a thin metal film (11), to the sensing layer (6a) attached on the other side of this metal film. The refraction of this beam changes in response to changes in the sensing layer (6a), and is usually measured as a change in the apparent reflection angle.

The sample (8) to which the prion sensor (10) is exposed may be a liquid sample, and may be buffered to maintain a pH that will not destroy the PrP^{C} or PrP^{Sc} proteins, or other components of the sample (8) that provide a measurable response. Many different buffers may be used and other agents may be included, such as detergents to suspend the proteins, protease inhibitors and anti-coagulants for blood samples. In one embodiment, the pH is maintained at about 7.4. As it has been hypothesized that a chaperone protein (Protein X described by Prusiner, 1998) may catalyse the conformational change of the prion protein from PrP^{C} to PrP^{Sc}, one embodiment of the method includes the addition of a chaperone protein to the sample (8), or to the sensing layer (6). The choice of this or any other additive to the sample (8) may be determined by the nature of the samples to be tested.

Without being limited to a theory, the response of the prion sensor may occur by several mechanisms, either individually or in combination. When PrP^{Sc} is present in sample (8), it may bind to the PrP^{C} sensing layer (6) causing a decrease in the resonant frequency, but no change in the motional resistance. This binding of PrP^{Sc} to the PrP^{C} sensing layer (6) may also initiate the conversion of the PrP^{C} to form new PrP^{sc}. Because the PrP^{C} molecule is soluble (hydrophilic) and the PrP^{Sc} molecule is insoluble (hydrophobic), the conversion of PrP^{C} molecules into PrP^{Sc} molecules on the surface of the sensor may cause the surface of the sensor to become more hydrophobic with time. As hydrophobic surfaces lose less acoustic energy than hydrophilic surfaces, the resonant frequency may increase while the motional resistance decreases (Hayward and Thompson, 1998). These resonance changes will occur at different times due to the different rates of the binding and/or conversion processes.

The ability of the method disclosed herein to diagnose TSE infection in a sample (8) may also be due to an interaction of the sensing layer (6) with components other than PrP^{Sc} in the sample, or which act with PrP^{Sc} in the sample to provide a measurable response.

When sample (8) contains cellular debris, the response may be more complex. PrP^{C} is normally present in the walls of nerve cells, held there by a glycosylphosphatidyl inositol (GPI) anchor. In the presence of cellular debris, sensing layer (6) may bind cell wall fragments without the interaction of PrP^{Sc}. This bound cellular debris may result in a mass and surface viscosity response, resulting in a decrease in the resonant frequency and an increase in the motional resistance. In addition to this background, the PrP^{Sc} in the sample may bind to the surface PrP^{C} providing a further decrease in the resonant frequency and increase in the motional resistance from increased acoustic coupling. The conversion of the sensing PrP^{C} to PrP^{Sc} may render it hydrophobic, decreasing the coupling and giving an opposing resonance change. These processes may occur at different rates, therefore an examination of the time course of the response may reveal the presence of PrP^{Sc}.

The sample (8) used herein may be prepared from various body tissues, such as brain, tonsil, eyelid, rectal and lymphatic tissue, or from bodily fluids such as blood, serum, urine and cerebrospinal fluid, or from bodily waste such as feces. The sample may be comprised of material from artificial tissue culture. The sample (8) may be a solution, suspension or emulsion that is prepared for example by homogenization, sonication, or other such tissue-disruption method. One or more additives may be added to the sample, such as for example, detergents, protease inhibitors and anti-coagulants.

Having thus described the various components of the prion sensor, the method of using the prion sensor to diagnose TSE, or to detect PrP^{Sc} molecules, in a sample will now be described.

In this method, a layer of PrP^{C} molecules alone or in combination with other molecules is attached to a surface of a sensor (20), to form a sensing layer (6) on the sensor. This sensing layer may be attached directly to the surface of the sensor, or via an intermediary linking layer (5). The sensing layer is then contacted with a sample from an animal suspected of having a TSE, or with a sample suspected of comprising PrP^{Sc} molecules. Methods of preparing the sample are discussed in the Examples, below.

An interaction between the sensing layer and a component of the sample is detected by determining whether there is an acoustic or optical response in the sensor, in response to the sample. This response is interpreted to diagnose TSE infection, or to detect the presence PrP^{Sc} molecules, in the sample. Whether there is an acoustic or optical response in the sensor may be determined by taking measurements while the sample is in contact with the sensing layer, and this is the preferred means of measuring this response. Alternatively, or in addition, the sample may be removed and additional new liquid may be contacted with the sensing layer and measurements may be taken. Alternatively again, the sample may be removed and the measurements may be taken with the sensing layer in a dry or relatively dry state.

If the prion sensor is made with a TSM sensor, the acoustic response in the TSM sensor with the attached layer of PrP^{C} molecules, may be measured by applying an electrical signal of known voltage, which is controlled at or scans through the series resonant frequency, to the acoustic sensor while it is in contact with the sample. The current through the sensor may also optionally be measured, to determine the motional resistance at the series resonant frequency in the sample. Alternately, the damping of the resonance may be measured to give the motional resistance. Changes in the series resonant frequency and/or the motional resistance are then interpreted to diagnose TSE infection or to detect the presence of PrP^{Sc} molecules in the sample, as shown in the Examples herein, or as known by persons of skill in the art.

If the prion sensor is made with a SPR sensor, the optical response of the SPR sensor with the attached layer of PrP^{C} molecules, may be measured by applying a beam of light to the sensor while in contact with the sample and the angle of refracted and reflected light may be measured. Changes in the angle of the reflected light is then interpreted to diagnose TSE infection, or to detect PrP^{Sc} molecules in the sample, using techniques known by persons of skill in the art.

The preferred method for interpretation is to quantify the difference between the measured response and a simple first order exponential model. Alternatively, there are many other possible numerical techniques that may be used to quantitatively interpret the data. Alternatively, qualitative interpretation may include recognizing features present in the response to infected samples relative to non-infected samples such as the shoulder for sheep brain samples and the frequency increase versus decrease in sheep urine samples. Non-infected controls are necessary for interpretation method development but are only necessary for quality control purposes in the method usage.

The method disclosed herein may be used to diagnose TSE, or to detect PrP^{Sc} molecules, using tissue samples such as brain, tonsil, rectal and eyelid, bodily fluids, such as urine, blood, cerebrospinal fluid and excrements such as feces. Further, the method may be used to detect PrP^{Sc} contamination in environmental samples such as soils.

While the invention has been described in conjunction with the disclosed embodiments, it will be understood that the invention is not intended to be limited to these embodiments. On the contrary, the invention is intended to cover alternatives, modifications and equivalents, which may be included within the spirit and scope of the invention as defined by the appended claims. Various modifications will remain readily apparent to those skilled in the art. Examples provided above and below are not intended to be limited to those examples alone, but are intended only to illustrate and describe the invention rather than limit the claims that follow.

### EXAMPLES

### Example 1

In this example the acoustic sensor was a TSM sensor made with a quartz crystal manufactured by Lap-Tech Inc. (Bowmanville, Ontario) with gold electrodes deposited onto both surfaces. The sensing layer was a recombinant sheep PrP^{C} protein commercially available (Roboscreen, Leipzig) linked to one of the gold electrodes by 11-mercapto undecanoic acid (11-MUA) activated by N-hydroxy succinimide (NHS) and 1-ethyl-3-(3-dimethyl aminopropyl) carbodiimide hydrochloride (EDC). This forms a peptide bond attaching the sensing layer (6) to the linking layer (5), which also is attached to the gold electrode (3) through the thiol group of 11-MUA.

More specifically, a quartz crystal was attached to the bottom of a cut off vial using silicone cement, as shown in FIG. 1, to create a cell to hold the sample (8). The gold electrode inside the vial (3) was cleaned with the following sequence of reagents: 10% nitric acid, water, acetone and ethanol, before coating with the mercaptan linker. The linking, activation and coating procedures were adapted from those used by Lyle et al. (2002). Under a nitrogen atmosphere the gold was coated with 11-MUA by soaking in a 10 mM solution of 11-MUA in ethanol at room temperature. After 24 hours, this solution was removed, the electrode was washed with ethanol, dried with nitrogen and capped.

At the time of each trial, one cell was opened and installed in the test fixture, which connected the electrodes (3 and 9) to the oscillator. 350 μL of 15 mM NHS was added to 350 μL of 75 mM EDC in a separate vial (at room temperature), mixed and then placed in the cell for 1 hour at 37° C. After this activation step, the activator solution was removed and the cell was washed with water. 200 μl of recombinant PrP^{C} solution, prepared by adding 1 mL of 10 mM acetate buffer at pH 4.0 to one 100 μg vial of protein as supplied by the manufacturer, was placed in the cell for 1 hour at 37° C. Each vial of recombinant PrP^{C} was sufficient for 5 trials. After this coating step the cell was washed with acetate buffer.

Each brain sample was prepared from a frozen homogenate that contained 350 mg of brain tissue in 1.75 mL of 5% glucose solution in 300 μL aliquots. 175 μL of this brain homogenate was added to 125 μL of buffer (Saborio et al., 2001) containing 0.5% Triton X-100 and 0.05% sodium dodecyl sulphate in phosphate buffered saline at pH 7.4. 50 μL of COMPLETE™ protease inhibitor (Roche Diagnostics GmbH, Germany) was also added at this step. This buffered homogenate was then mixed, added to the cell and data was collected for at least 4 hours, with the cell incubated at 37° C.

The series resonant frequency and motional resistance were measured using a Maxtek, Inc. (Santa Fe Springs, Calif.) PLO-10 phase-locked oscillator. The motional resistance data were found to follow the same trends as the series resonant frequency. While only the frequency response is discussed below, it is understood that the same information regarding the determination of infectious PrP^{Sc} may be obtained from the motional resistance data.

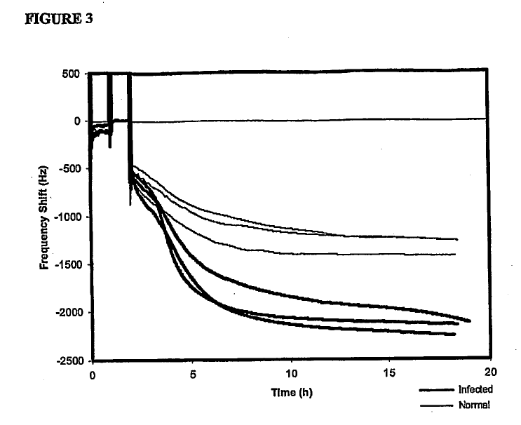

FIG. 3 shows the frequency response of this sensor to both TSE infected and normal sheep brain homogenates. The first hour of the response was the activation of 11-MUA coated crystals by a mixture of NHS and EDC. The second hour is the deposition of the sensing protein, recombinant sheep PrP^{C}. After the sensing layer (6) was formed, the crystal wafer was rinsed and the brain homogenate was applied. In response to the normal tissue sample, the sensor detected a drop in frequency, which was likely due to the attachment of cellular debris. In response to the infected tissue sample, the sensor ultimately detected an unmistakably larger frequency drop, perhaps due to the additional binding capacity between the sensing PrP^{C} and the infected PrP^{Sc}. More important, however, is the shoulder at the beginning of the response observed with the “infected” sample, which is perhaps caused by the conversion of the surface-attached PrP^{C} to PrP^{Sc}, giving a decreased acoustic coupling, and resulting in a lowered rate of frequency decrease.

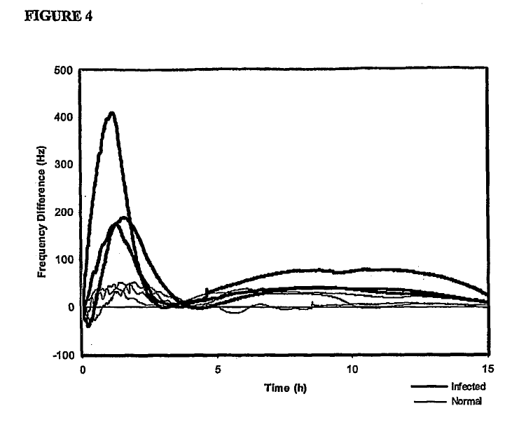

This observed shoulder may be exploited as the main indicator of infection as shown in FIG. 4. The method of interpretation used here is based on the difference between the measured response and a simple first order exponential response model, although there are many other possible numerical techniques that may be used to process the data. The peak showing the size of the shoulder is absent for the normal samples but is pronounced for the infected samples.

This example confirms that this assay system is rapid, sensitive and technically simple. The assay is rapid as the peaks shown in FIG. 4 were complete after only two hours after placing the sample in the test apparatus. The assay is technically simple, as it requires no protease treatment to destroy normal PrP^{C}. No pretreatment other than homogenization was required.

In a manner similar to the above, samples of eyelid, tonsil, rectal and lymphatic tissue could also be prepared and assayed. There may be some differences as to how the samples are prepared, compared to the preparation described above, for example these tissues may require the use of different buffers or stabilizing agents, a different means of tissue disruption, or a different dilution of the sample, but these variations could be determined by someone of skill in the art with routine testing.

In a manner similar to the above, samples from other species may be tested with PrP^{C} from that same species.

In a manner similar to the above, fresh samples may be used.

### Example 2

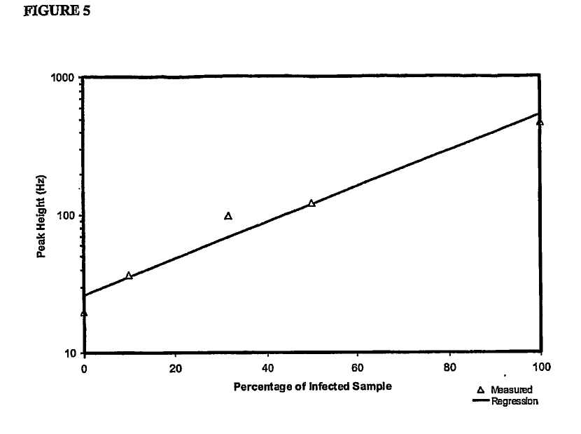

Infected sheep brain homogenate was diluted with homogenate from uninfected brain samples, and subjected to the procedure outlined above, to determine whether the method is quantitative. The height of the observed peak determined as above is logarithmically proportional to the infected homogenate concentration, and therefore possibly also the PrP^{Sc} concentration, as shown by the dilution series data in FIG. 5. This confirms that the assay provides quantitative data, as the peak heights of FIG. 5 were dependent on the amount of infected homogenate in the sample that, was assayed.

### Example 3

The method disclosed herein may be used to diagnose TSE or to detect PrP^{Sc} molecules in mammals other than sheep. The example provided above may be repeated using brain homogenates from elk that exhibit symptoms of chronic wasting disease (CWD) and normal (CWD-free) elk, to demonstrate that the method will work in different mammalian species, and therefore is not limited to the detection of Scrapie in sheep. Frozen brain samples of diseased and non-diseased animals were prepared, as described above for the samples of sheep brain. The same assay procedure as outlined above was used. The sensing layer was the same, consisting of recombinant normal sheep PrP^{C} molecules attached in the same manner.

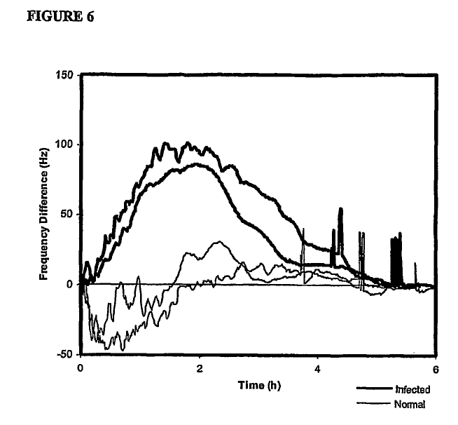

FIG. 6 shows the results, the same in form as those obtained from sheep with Scrapie. The ability of the assay to diagnose CWD in elk is clearly shown.

In a manner similar to the above, TSE's in additional species, beyond sheep and elk, may be diagnosed.

### Example 4

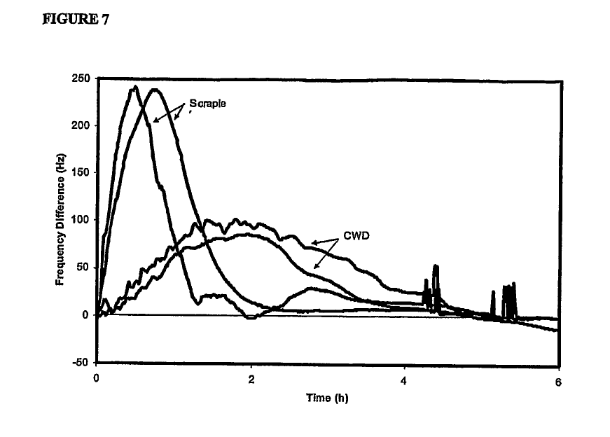

The method disclosed herein may be used across a species barrier to further provide a quick and easy assessment of cross-species vulnerability to infection by prions. Cross-species detection (and infectivity) likely requires the sample (8) from an infected animal, or the PrP^{Sc} molecule therein, to have the ability to trigger a conformational or other change of, or interact with, normal PrP^{C} from an animal of a different species, that is immobilized in the sensing layer. This cross-species detection of prion infection has been demonstrated in the previous example. FIG. 7 shows the response of a TSM sensor coated with sheep PrP^{C} exposed to brain samples from elk that have CWD and to brain samples from sheep that have Scrapie. The sheep PrP^{C} sensing layer is sensitive to both, however the response of the sensor was slower or lower in the cross-species situation, and therefore was less efficient than the response to the sample from the same species. The method may, therefore, be used as a measure of cross-species infectivity, and cross-genotype infectivity.

### Example 5

In this example, urine samples were assayed by the above outlined method. 15 mL samples of urine from sheep with Scrapie and normal sheep, was concentrated by ultrafiltration. The filter passed molecules up to 5000 Daltons in size, and therefore the prion proteins were held in the retentate, which was concentrated to a volume of 175 μL. This was added to the buffer and protease inhibitor, as above, and subjected to the same assay as discussed above.

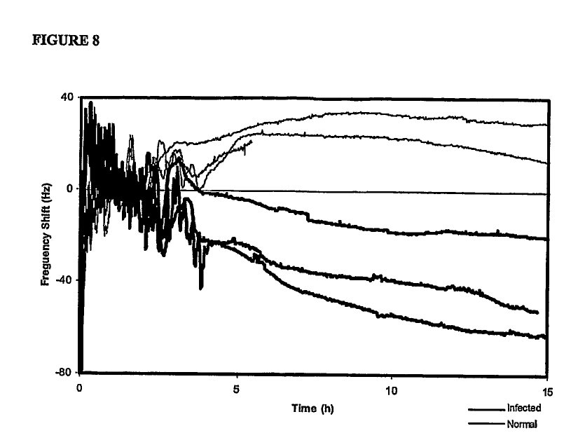

FIG. 8 shows the frequency response of this sensor to the urine sample from normal sheep and sheep with Scrapie. The response is different from that obtained with samples from brain. This is likely due to the smaller amount of cellular debris in the sample. The data clearly shows the enhanced interaction of the sensor with the Scrapie samples.

In a manner similar to the above, samples of other bodily fluids could also be prepared and assayed. There may be some differences in how the samples are prepared, as compared to the preparation described above, for example these fluids may require the use of different buffers or stabilizing agents, a different means of filtering the sample to remove contaminants, or dilution rather than concentration, but these variations could be determined by someone of skill in the art with routine testing.

### REFERENCES

The following references are cited in the application at the relevant portion of the application. Each of these references is incorporated herein by reference.
Borman (1998) Chem. and Engng. News, February 9: 22-29.
Borman (2001) Chem. and Engng. News, April 9: 38-39.
Bruce (2003) British Medical Bulletin, 66:99-108.
Cavic et al. (1997) Faraday Discuss. 107: 159-176.
Collinge (2001) Annu. Rev. Neurosci. 24:519-550.
Ferrante et al. (1994) J. Appl. Phys. 76: 3447-3462.
Hayward and Thompson (1998) J. Appl. Phys. 83: 2194-2201.
Kipling and Thompson (1990) Anal. Chem. 62: 1514-1519.
Lyle, E-L. et al., (2002) Analyst, 127: 1596-1600.
Prusiner (1996) Prions, Prions, Prions in Human Prion Diseases and Neurodegeneration, Prusiner, S. B. (Ed), Springer Verlag, Berlin.
Prusiner, S. B., Williams, E., Laplanche, J-L. and Shinagawa, M. Scrapie, Chronic Wasting Disease and Transmissible Mink Encephalopathy, in Prion Biology and Diseases, S. B. Prusiner, ed., Cold Spring Harbor Laboratory Press, Cold Spring Harbor, N.Y., 2004.
Prusiner (1998) Proc. Natl. Acad. Sci. USA 95: 13363-13383.
Pan, et al. (1993) Proc. Natl. Acad. Sci. USA 90: 10962-10966.
Saborio et al. (2001) Nature 411: 810-813.
Su and Thompson (1996) Can. J. Chem. 74: 344-358.

1. A method of diagnosing TSE, or of detecting PrP^{Sc} molecules, in a sample, which method comprises:
(a) attaching PrP^{C} molecules to a surface of a sensor directly, or via a linking layer, to form a sensing layer;
(b) contacting the sensing layer with the sample;
(c) measuring an acoustic or optical response in the sensor, and
(d) interpreting the response of the sensor, to diagnose TSE, or to detect PrP^{Sc} molecules, in the sample.

2. The method of claim 1, wherein the sensor is an acoustic sensor, and the acoustic response is determined by:
(a) inducing oscillating motion at the acoustic resonant frequency of the sensor;
(b) measuring this resonant frequency, or alternately measuring the energy loss as:
(i) a motional resistance, or
(ii) a dissipation factor, and
(c) interpreting the measurement of the resonant frequency, the motional resistance and/or the dissipation factor, to diagnose TSE, or to detect PrP^{Sc} molecules, in the sample.

3. The method of claim 2, wherein the acoustic sensor is a TSM sensor.

4. The method of claim 1, wherein the sensor is an optical sensor, and the optical response is determined by:
(a) applying an optical beam to the sensor;
(b) measuring the refraction and/or reflection characteristics of the sensor;
(c) interpreting the measurement of the refraction and/or reflection characteristics, to diagnose TSE, or to detect PrP^{Sc} molecules, in the sample.

5. The method of claim 4, wherein the optical sensor is an SPR sensor.

6. The method of claim 1, wherein the step of measuring the acoustic or optical response of the sensor is performed while the sensor or the sensing layer is in contact with the sample.

7. The method of any of claims claim 1 to 6, further comprising the step of adding a chaperone protein or a tissue extract comprising a chaperone protein to the sample before or during step (b).

8. The method of claim 1, wherein more than one layer of PrP^{C} molecules is attached to the surface of the sensor, to form the sensing layer.

9. The method of any of claim 1, wherein the sample is from a mammal.

10. The method of claim 9, wherein the mammal is selected from the group consisting of: sheep, deer, cow, human, mink, hamster, mouse, goat and cat.

11. The method of claim 9, wherein the sample is comprised of an extract of a tissue from the mammal.

12. The method of claim 11 wherein the tissue is selected from the group consisting of: tonsil, rectal, eyelid, brain and lymphatic tissue.

13. The method of claim 12 wherein the tissue is brain.

14. The method of claim 9 wherein the sample is comprised of bodily fluid from a mammal.

15. The method of claim 14 wherein the bodily fluid is selected from the group consisting of: blood, serum, urine and cerebrospinal fluid.

16. The method of claim 15 wherein the sample is comprised of urine.

17. The method of claim 9 wherein the sample is comprised of excrement.

18. The method of claim 1, wherein the sample is comprised of material from an artificial tissue culture.

19. The method of claim 1, wherein the sample comprises material obtained from environmental sampling.

20. The method of claim 1, wherein the sample and the PrP^{C} molecule are from the same mammalian species.

21. The method of claim 1 wherein the sample and the PrP^{C} molecule are from different genotypes of the same mammalian species.

22. The method of any of claim 1, wherein the sample and the PrP^{C} molecule are from different mammalian species.

23. The use of PrP^{C} as a sensing molecule in an acoustic or optical measuring device that can detect molecular changes in the sensing molecule, to diagnose TSE, or to detect PrP^{Sc} molecules in a sample.

24. The use in claim 23, wherein the PrP^{C} is isolated from healthy animal tissue, produced by a recombinant host organism or artificially synthesized.

25. The use in claim 23, wherein the acoustic device is a thickness shear mode piezoelectric oscillating molecular sensing device.

26. The use in claim 23, wherein the optical device is a surface plasmon resonance device.

27. A prion sensor comprising an acoustic sensor and a layer of PrP^{C} molecules attached directly or indirectly to a surface of the acoustic sensor.

28. The prion sensor of claim 27, wherein the acoustic sensor is a TSM sensor.

29. A prion sensor comprising PrP^{C} molecules attached directly or indirectly to a surface of an optical sensor.

30. The prion sensor of claim 29, wherein the optical sensor is an SPR sensor.

31. A method of quantitating the amount of PrP^{Sc} in a sample, which method comprises practicing the method claimed in claim 1 and interpreting the response in the series resonant frequency, the motional resistance, the acoustic dissipation factor, or the refraction and/or reflection characteristics of the optical beam, to determine the amount of PrP^{Sc} in the sample.

32. A method of measuring the capability of PrP^{Sc} from one species to infect a different species, which method comprises practicing the method claimed in claim 1, characterized in that the PrP^{C} molecules and the PrP^{Sc} molecules are from different species.

33. A method of measuring the capability of PrP^{Sc} from one genotype of a species to infect a different genotype of the same species, which method comprises practicing the method claimed in claim 1, characterized in that the PrP^{C} molecules and the PrP^{Sc} molecules are from different genotypes of the same species.
